(19) United States
(12) Patent Application Publication
(10) Pub. No.: US 2025/0210162 A1
(43) Pub. Date: Jun. 26, 2025

(54) SYSTEM AND METHOD FOR MANAGING AND MONETIZING ORAL HEALTH DATA ON A BLOCKCHAIN-BASED PLATFORM

(71) Applicants: Paul Andrew Dominique, Atlanta, GA (US); Mina Amir Youssef, Mableton, GA (US)

(72) Inventor: Paul Andrew Dominique, Atlanta, GA (US)

(21) Appl. No.: 19/063,383

(22) Filed: Feb. 26, 2025

(51) Int. Cl. G16H 10/60; G06Q 30/0207; G06Q 30/0601; H04L 9/00

(52) U.S. Cl. CPC G16H 10/60; G06Q 30/0224; G06Q 30/0631; H04L 9/50; H04L 2209/56

(57) ABSTRACT

A system for a blockchain-based platform that integrates oral health monitoring, personalized preventive care, and data monetization. The system for blockchain-based platform comprises a computing device that has a processor and a memory for storing one or more instructions executable by the processor. The computing device is in communication with a server via a network. The system is designed to reduce dental healthcare expenses by promoting preventive care and enabling the monetization of the oral health data, and the clinical-grade data. The system facilitates user ownership, transparency, and control over personal dental health records, offering options for users to monetize the oral health data, and the clinical-grade data. The system integrates the subscriber-controlled digital wallet to ensure guaranteed oral health services.

### FIELD OF INVENTION

The present disclosure relates generally to preventive dental care, data analytics, and blockchain technology, and more particularly relates to a system and method for integrating oral health monitoring, personalized preventive care, and oral health data monetization on a blockchain-based platform.

### BACKGROUND

Orthodontics is a branch of dentistry that deals with managing of irregularities related to alignment of teeth, malocclusions, smile correction and other structural and aesthetic deformities related to teeth and smile of a patient. Currently orthodontic care is managed by a professional care provider which is reactive and craft based. This process adds substantially to the cost of care and leads to poor care outcomes. Provision of quality orthodontic care involves focus on certain key dimensions, such as patient centeredness, access to care, efficient therapeutics, patient safety, and clinical effectiveness.

Dental health is a critical aspect of overall well-being, with preventive care playing a vital role in avoiding costly and invasive treatments. Despite advances in oral hygiene awareness, many individuals still face significant barriers in accessing affordable preventive care, maintaining consistent oral health habits, and utilizing their health data effectively.

The current dental care system is heavily reactive, focusing on therapeutic treatments like fillings and crowns rather than preventive measures. This results in high treatment costs and limited emphasis on early disease detection and prevention. Furthermore, patients lack control over their oral health data, which remains underutilized for generating value or improving access to personalized care.

Technologies such as smart toothbrushes, saliva testing kits, and dental apps have emerged to monitor oral health metrics, offering insights into brushing habits, plaque levels, and microbiological profiles. Blockchain-based platforms and data marketplaces have also gained traction in healthcare, enabling patients to manage and share medical data securely.

However, these solutions are fragmented and lack integration into a unified system. Smart devices and apps often provide isolated insights without actionable guidance or incentives for users. Blockchain platforms, while secure, have not yet been applied effectively to dental health. Additionally, there is no established framework to monetize dental data or oral health data and enable group-based negotiations for better care plans, leaving patients with limited leverage in the dental industry.

Therefore, there is a need for a system that integrates preventive care, data ownership, and monetization into a single, patient-centric ecosystem. There is also a need for a system and method for integrating oral health monitoring, personalized preventive care, and oral health data monetization on a blockchain-based platform.

### SUMMARY OF THE INVENTION

The following presents a simplified summary of one or more embodiments of the present disclosure to provide a basic understanding of such embodiments. This summary is not an extensive overview of all contemplated embodiments and is intended to neither identify key nor critical elements of all embodiments, nor delineate the scope of any or all embodiments.

The present disclosure, in one or more embodiments, relates to a system for a blockchain-based platform that integrates oral health monitoring, personalized preventive care, and data monetization.

An embodiment of a first aspect wherein the system for blockchain-based platform comprises a computing device that has a processor and a memory for storing one or more instructions executable by the processor. The computing device is in communication with a server via a network.

In one embodiment, the processor is configured to enroll one or more subscribers in a dental plan through a subscription management module. A data collection module is configured to receive dental data or oral health data of the one or more subscribers associated with one or more dental activities. The data collection module comprises one or more dental devices that include at least one of a smart toothbrush attachment, a saliva testing kit, and a plaque scoring unit. The one or more dental activities are performed by the dental device. The oral health data comprises brushing data, saliva data, and plaque scoring data.

In one embodiment, an artificial intelligence (AI) module is configured to analyze the oral health data to generate a custom dental health score, thereby determining schedule for one or more hygiene appointments to maintain oral health based on the custom dental health score. The AI module is configured to provide recommendations for personalized oral hygiene products based on the custom dental health score.

In one embodiment, a clinical data collection module is configured to obtain clinical-grade data of the one or more subscribers during the one or more hygiene appointments. The clinical-grade data comprises at least one of intraoral radiographs, cone beam computed tomography (CBCT) scans, and 3D intraoral scans, that is collected during the one or more hygiene appointments.

In one embodiment, a marketplace module is configured to enable the one or more subscribers to upload the clinical-grade data to a marketplace. The marketplace module is further configured to enable the one or more subscribers to combine the oral health data with financial credit scores to enhance a market value on the marketplace. The marketplace module is further configured to enable the one or more subscribers to monetize and aggregate the oral health data into collective pools for group negotiations with one or more third-party service providers to secure desired pricing or dental plans, either individually or as a group. The marketplace module is configured enable the one or more subscribers to select specific buyers or industries for monetizing the collected oral health data, and the clinical-grade data.

In one embodiment, a reward module is configured to issue one or more tokens when the one or more subscribers perform at least one activity that comprises sharing the custom dental health score, and regularly attending the one or more hygiene appointments. The reward module is configured to generate new tokens based on compliance with the dental plan. The reward module is configured to enable trading of the one or more tokens on a cryptocurrency exchange, providing liquidity to the one or more subscribers. The one or more tokens are configured to be used for discounts on dental care or related products within the blockchain-based platform.

In one embodiment, one or more databases are configured for storing the collected oral health data, the clinical-grade data, and the custom dental health score of the one or more subscribers. The one or more databases are accessible through a subscriber-controlled digital wallet. The one or more databases comprise at least one blockchain-enabled database. The subscriber-controlled digital wallet is configured to integrate a visualization dashboard that displays a subscriber's oral health progress and compliance history.

In one embodiment, the server is configured to execute gamified compliance programs, awarding the one or more tokens to the one or more subscribers for maintaining healthy dental metrics.

An embodiment of a second aspect wherein a method for monetizing dental data or oral health data is disclosed. At first step, the oral health data of the subscribers associated with the dental activities is received from the data collection module. Next, the oral health data is analyzed by the AI module to generate the custom dental health score, thereby determining schedule for the hygiene appointments to maintain oral health based on the custom dental health score. Next, the clinical-grade data of the subscribers is obtained by the clinical data collection module during the hygiene appointments. Next, the subscribers are enabled to upload the clinical-grade data to the marketplace by the marketplace module.

In one embodiment, next, the subscribers are enabled to combine the oral health data with financial credit scores to enhance the market value on the marketplace through the marketplace module. Next, the subscribers are enabled to monetize and aggregate the oral health data by the marketplace module. The aggregated data is collected into collective pools for group negotiations with third-party service providers. These negotiations aim to secure desired pricing or dental plans, where the negotiations can occur either individually or as a group.

In one embodiment, next, the tokens are issued to the subscribers, by the reward module, upon performing at least one activity that comprises, but is not limited to, sharing the custom dental health score, and regularly attending the hygiene appointments. Later, the collected oral health data, the clinical-grade data, and the custom dental health score are stored in the databases accessible through the subscriber-controlled digital wallet.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

The accompanying drawings, which are incorporated in and constitute a part of the specification, illustrate an embodiment of the invention, and, together with the description, explain the principles of the invention.

FIG. 1 illustrates a block diagram of a system for a blockchain-based platform to manage and monetize oral health data, in accordance with embodiments of the invention.

FIG. 2 illustrates a flowchart of a method for monetizing oral health data using the system, in accordance with embodiments of the invention.

### DETAILED DESCRIPTION

Reference will now be made in detail to the present preferred embodiments of the invention, examples of which are illustrated in the accompanying drawings. Wherever possible, the same reference numerals are used in the drawings and the description to refer to the same or like parts.

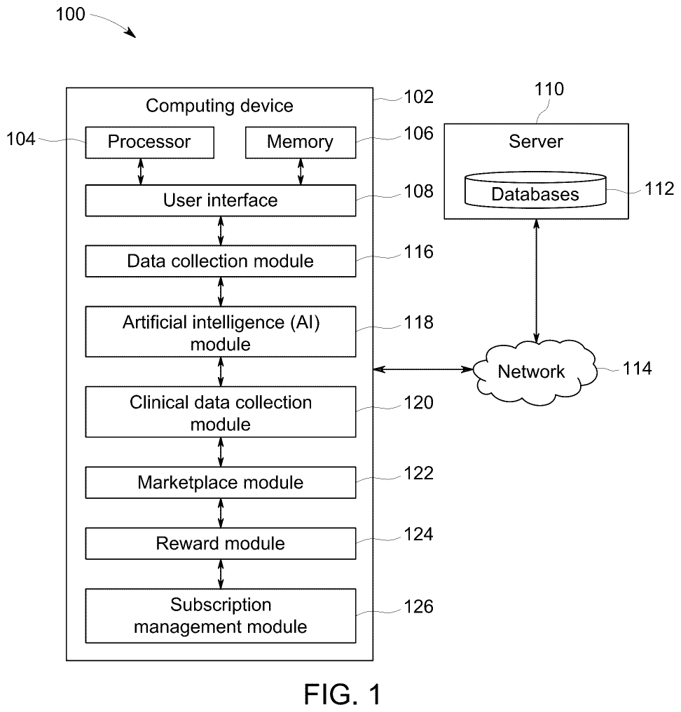

FIG. 1 refers to a block diagram of a system 100 for a blockchain-based platform to manage and monetize oral health data. In one embodiment herein, the system 100 provides a secure and transparent framework for storing, organizing, and sharing oral health information, such as clinical records, imaging, and test results on the blockchain-based platform. By leveraging blockchain technology, it ensures data integrity, security, and controlled access, empowering users to have complete control over the oral health data. Additionally, the system 100 incorporates mechanisms for monetization, enabling the users to generate revenue by sharing their oral health data with authorized stakeholders, such as researchers, healthcare providers, and businesses. Through tokenized transactions and smart contracts, the system 100 streamlines data exchanges while incentivizing the users, creating an ecosystem that integrates technological innovation with economic benefits.

In one embodiment herein, the system 100 comprises a computing device 102 having a processor 104 and a memory 106, which stores one or more instructions executable by the processor 104. These instructions may be executed to cause the system 100 to perform the various functionalities. The computing device 102 is in communication with a server 110 via a network 114.

In one embodiment, the processor 104 acts as the central processing unit (CPU) of the system 100, responsible for coordinating different tasks and carrying out complex operations, data processing, and decision-making by fetching instructions from the memory 106, thereby decoding the instructions and executing the necessary actions.

In one embodiment, the processor 104 is configured to enroll one or more subscribers in a dental plan. The dental plan combines blockchain technology with data-driven analytics to provide the subscribers with guaranteed oral health outcomes while enabling privacy, and transparency. The system 100 comprises one or more databases 112 that are configured to store user data of the subscribers. The databases 112 are accessible through a subscriber-controlled digital wallet. The databases 112 comprise at least one blockchain-enabled database.

In one embodiment, the user data comprises, but is not limited to, subscriber dental medical records. Further, the subscriber-controlled digital wallet is self-executing agreements with terms written directly into code.

In one embodiment herein, the memory 106 serves as the storage component of the system 100, holding the executable instructions, as well as any data or information required by the processor 104 to perform its tasks. The data includes user inputs, system configurations, and any other relevant data needed for the system's operations. Through the communication between the processor 104 and the memory 106, the system 100 is able to process the user inputs, access stored information, perform computations, and make decisions accordingly.

In one embodiment herein, the computing device 102 represents any electronic device that each subscriber can utilize to interact with the system 100. The computing device 102 can be, but not limited to, a smartphone, a laptop, a tablet, a personal computer, or any other suitable electronic device. The computing device 102 serves as the user's gateway to accessing and interacting with the system 100. The computing device 102 is configured to enable the subscriber to engage with the system's functionalities and capabilities through a user interface 108.

In one embodiment herein, the user interface 108 is a crucial component of the computing device 102, which allows the subscribers to input commands, receive information, and control the system 100. The user interface 108 can be, but not limited to, a touch screen, a keyboard, a mouse, voice recognition modules, gesture recognition sensors, and virtual reality interfaces. The versatility of the user interface 108 ensures that the subscribers can engage with the system 100 in a manner that is most intuitive and comfortable for the subscribers, thereby catering to a wide range of subscriber's preferences and accessibility needs. The computing device 102 empowers the subscribers to interact with the system 100 seamlessly and efficiently by providing multiple user interface options, thereby leveraging the most appropriate input and output modalities for their specific needs and preferences.

In one embodiment herein, the computing device 102 is in communication with the server 110 and the databases 112 via the network 114. The network 114 acts as a communication that allows the computing device 102 to interact with the other components of the system 100, thereby facilitating the exchange of data, commands, and information. In one embodiment herein, the network 114 can be a wireless communication infrastructure, which offers the subscribers flexibility and convenience when interacting with the system 100. This wireless connectivity enables the subscribers to access the system 100 from various locations, without being tethered to a fixed physical connection.

In one embodiment herein, the network 114 can be, but not limited to, Local Area Network (LAN), Cellular Network, Wide Area Network (WAN), Intranet, Virtual Private Network (VPN), and wireless networks that use radio frequency (RF) or infrared (IR) technology to transmit data without the need for physical cables, thereby providing mobility and flexibility. The versatility of the network 114 ensures that the computing device 102 can seamlessly connect to the server 110 and the databases 112, thereby enabling the subscribers to access the system's 100 functionalities and resources from a variety of locations and devices. This wireless connectivity enhances the overall accessibility and convenience of the system 100 for the subscribers.

In one embodiment, the system 100 comprises a data collection module 116, an artificial intelligence (AI) module 118, a clinical data collection module 120, a marketplace module 122, a reward module 124, and a subscription management module 126.

In one embodiment, the data collection module 116 is configured to receive dental data or oral health data of the subscribers associated with one or more dental activities. The data collection module 116 comprises one or more dental devices that include at least one of a smart toothbrush attachment, a saliva testing kit, and a plaque scoring unit. The dental activities are performed by the dental device. The oral health data comprises brushing data, saliva data, and plaque scoring data.

In one embodiment, the smart toothbrush attachment logs brushing habits of the subscribers in the databases 112. The saliva testing kit identifies pathogenic bacteria or oral health risks (e.g., cavities or gum disease potential). The plaque scoring data provides insights into the effectiveness of the subscriber's brushing technique.

In another embodiment, the oral health data may be collected through the user interface 108, which facilitates collection of the oral health data in a form, a questionnaire, a self-help portal, a website-based subscriber registration and the like. In some example embodiments, the user interface 108 may also include an option to capture or scan or upload an image to identify subscriber's anatomical features, such as subscriber's bone face structure, teeth geometry, gums geometry and other relevant attributes.

In one embodiment, the AI module 118 is configured to analyze the oral health data to generate a custom dental health score, thereby determining schedule for one or more hygiene appointments to maintain oral health based on the custom dental health score. The AI module 118 is configured to provide recommendations for personalized oral hygiene products based on the custom dental health score.

In another embodiment, the oral health data comprises dental structures of the subscribers. The AI module 118 is configured to analyze the oral health data for assessing subscriber's health risks, diagnoses, and potential treatment recommendations personalized based on the custom dental health score. The AI module 118 is configured to analyze plaque patterns based on the plaque scoring data. The plaque patterns provides insights into the effectiveness of the subscriber's brushing technique. The AI module 118 is further configured guide the subscribers on hygiene practices and product selection.

In one embodiment, the clinical data collection module 120 is configured to obtain clinical-grade data of the subscribers during the hygiene appointments. The clinical-grade data comprises, but is not limited to, intraoral radiographs, cone beam computed tomography (CBCT) scans, and 3D intraoral scans, that is collected during the hygiene appointments.

In one embodiment, the marketplace module 122 is configured to enable the subscribers to upload the clinical-grade data to a marketplace. The marketplace module 122 is further configured to enable the subscribers to combine the oral health data with financial credit scores to enhance a market value on the marketplace.

In one embodiment, the marketplace module 122 is configured to enable the subscribers to monetize and aggregate the oral health data into collective pools for group negotiations with one or more third-party service providers to secure desired pricing or dental plans, either individually or as a group. The marketplace module 122 is configured to empower the subscribers to unlock the value of the oral health data through monetization and collective bargaining. Specifically, the data collection module 116 allows the subscribers to upload and manage the oral health data and the clinical-grade data securely within the blockchain-based platform.

In one embodiment, the marketplace module 122 is configured enable the subscribers to select specific buyers or industries for monetizing the oral health data, and the clinical-grade data. The marketplace module 122 allows the subscribers to monetize the oral health data and the clinical-grade data directly by selling it to the third-party service providers that comprise, but are not limited to, dentists, research institutions, pharmaceutical companies, or healthcare organizations, in exchange for financial compensation or other benefits.

In one embodiment, the marketplace module 122 is enables the subscribers to aggregate the oral health data, and the clinical-grade data with other subscribers, forming the collective data pools. The collective data pools create stronger bargaining power, allowing groups of subscribers to negotiate collectively with the third-party service providers for better deals, such as reduced pricing on dental treatments, enhanced care packages, or group discounts. This feature also allows the third-party service providers to efficiently target larger groups of potential patients or data sets, increasing the overall value of participation for both parties.

In one embodiment, the marketplace module 122 allows the subscribers to select to engage individually, monetizing respective oral health data, and respective clinical-grade data for personal benefit, or participate in group negotiations to leverage collective influence. This functionality ensures flexibility while maximizing opportunities for both individual and collective gains, creating a more dynamic and patient-driven oral health data marketplace.

In an exemplary embodiment, the marketplace module 122 is enables the subscribers to monetize the oral health data and the clinical-grade data through the marketplace. For data monetization the subscribers are allowed to upload the oral health data and the clinical-grade data to the marketplace. In one embodiment, the buyers include, but are not limited to, dentists, dental professionals, researchers, or other buyers. The buyers bid or pay a price to access the oral health data and the clinical-grade data of at least one subscriber or a group of subscribers. Pricing depends on factors such as the quality and depth of the oral health data and the clinical-grade data. The subscriber's collective data pools with other subscribers. Additional data enhancements, such as combining dental health metrics with the financial credit scores.

In another embodiment, the dentists may offer competitive pricing or bids to provide services to subscribers based on their data and financial profiles. In one embodiment, the marketplace is configured to revolutionize lead generation for dentists. The marketplace provides dentists the ability to select cases based on their preferences, skills, or revenue goals. The flexibility of the system 100 that allows the dentists select preferred cases disrupts the traditional clinic model, allowing dentists to manage patients (subscribers) without owning a physical office by leveraging short-term or one-time rentals and online recruitment.

In one embodiment, the dentists are granted access to a subscriber's complete dental record upon payment of a competitive fee. By enabling seamless and secure retrieval of dental history, the system 100 allows dental professionals to make well-informed treatment decisions, minimizing redundant diagnostic procedures and enhancing the efficiency of dental care. Furthermore, the system 100 facilitates a subscriber's transition between different dental clinics or providers, reducing administrative burdens and improving the overall patient experience.

In one embodiment, the reward module 124 is configured to issue one or more tokens when the subscribers perform at least one activity that comprises sharing the custom dental health score, and regularly attending the hygiene appointments. The reward module 124 is configured to generate new tokens based on compliance with the dental plan. The tokens are awarded when the subscribers allow their oral health data to be shared with the system 100 or marketplace.

The reward module 124 is configured to enable trading of the tokens on a cryptocurrency exchange, providing liquidity to the subscribers. The tokens are configured to be used for discounts on dental care or related products within the blockchain-based platform.

In one embodiment, the server 110 is configured to execute gamified compliance programs, and awarding the tokens to the subscribers for maintaining healthy dental metrics through the reward module 124. The tokens are earned for following preventive care schedules, such as attending prescribed hygiene appointments or maintaining healthy dental metrics such as brushing frequency, plaque scores.

In one embodiment, the subscription management module 126 is configured to enroll subscribers in a dental plan requiring compliance with data sharing and preventive appointments. The subscription management module 126 is configured to implement a guarantee for oral health outcomes. The subscription management module 126 prevents upselling of therapeutic dental treatments during preventive hygiene appointments.

In one embodiment, the subscriber-controlled digital wallet ensures the payment of restorative treatment costs for cavities in virgin teeth if the subscriber complies with the plan. The subscriber-controlled digital wallet is configured to store subscriber data, and allow the subscribers to control, manage, and share the oral health data, and the clinical-grade data.

In one embodiment, the databases 112 are configured for storing the collected oral health data, the clinical-grade data, and the custom dental health score of the subscribers. The subscriber-controlled digital wallet is configured to integrate a visualization dashboard that displays a subscriber's oral health progress and compliance history. In one embodiment herein, the subscriber-controlled digital wallet governs the interaction between the system 100 and the subscribers. The subscribers agree to share the oral health data (such as brushing frequency, plaque scores, saliva microbiome results) collected through the dental devices. The subscribers agree to attend a prescribed number of the hygiene appointments determined by the AI module 118 derived from the oral health data.

In another embodiment, the system 100 for associating one or more blockchain enabled databases 112 with the blockchain-based platform may be provided. The blockchain enabled, encrypted, secured and tamper-proof databases 112 have security, hierarchical levels of access to various agents, time stamped data records, such as subscriber dental medical records which may be speech, text, image, video, for providing one or more but not limited to care outcomes, product performance, patient adherence, learning, performance analytics and training recommendations.

In some embodiments, the system 100 is designed to offer a paid, continuous health monitoring service for the subscribers, ensuring ongoing tracking and management of the oral health data of the subscribers.

In some embodiments, the system 100 utilizes the databases 112 integrated with the blockchain-based platform to ensure the security and integrity of sensitive dental medical records. The blockchain-based platform leverages advanced cryptographic techniques, including private and public keys, to safeguard the data. The use of blockchain ensures that once data is recorded, it cannot be altered or tampered with, providing an immutable and transparent record of the subscriber's dental history.

In some embodiments, the private keys enable restricted access to the subscriber's data, ensuring that only authorized personnel such as the subscribers are allowed to view or modify the records. The public keys, on the other hand, are used for validation purposes, allowing or approved buyers to confirm the authenticity and integrity of the records without compromising privacy. This combination of blockchain technology and key-based security protocols provides a highly secure environment for the storage, transfer, and access of dental medical records, fostering trust and confidentiality in the system.

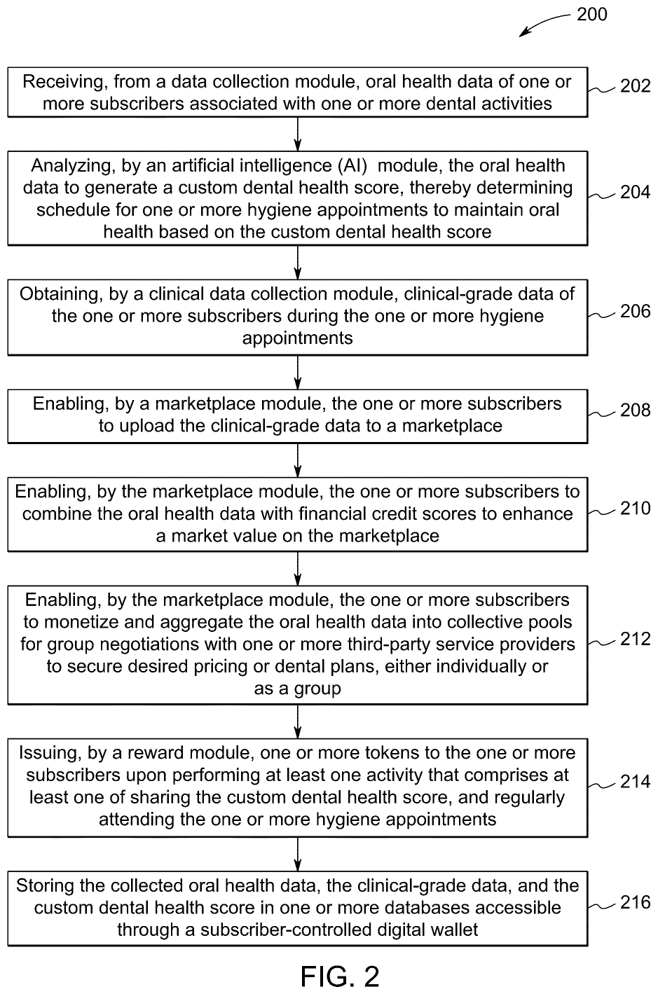

FIG. 2 refers to a flowchart 200 of a method for monetizing oral health data is disclosed. At step 202, the oral health data of the subscribers associated with the dental activities is received from the data collection module 116. At step 204, the oral health data is analyzed by the AI module 118 to generate the custom dental health score, thereby determining schedule for the hygiene appointments to maintain oral health based on the custom dental health score. At step 206, the clinical-grade data of the subscribers is obtained by the clinical data collection module 120 during the hygiene appointments. At step 208, the subscribers are enabled to upload the clinical-grade data to the marketplace by the marketplace module 122.

In one embodiment, at step 210, the subscribers are enabled to combine the oral health data with financial credit scores to enhance the market value on the marketplace through the marketplace module 122. At step 212, the subscribers are enabled to monetize and aggregate the oral health data by the marketplace module 122. The aggregated data is collected into collective pools for group negotiations with third-party service providers. These negotiations aim to secure desired pricing or dental plans, where the negotiations can occur either individually or as a group.

In one embodiment, at step 214, the tokens are issued to the subscribers, by the reward module 124, upon performing at least one activity that comprises, but is not limited to, sharing the custom dental health score, and regularly attending the hygiene appointments. At step 216, the collected oral health data, the clinical-grade data, and the custom dental health score are stored in the databases 112 accessible through the subscriber-controlled digital wallet.

In one embodiment, the system 100 is designed to reduce dental healthcare expenses by promoting preventive care and enabling the monetization of the oral health data, and the clinical-grade data. The system 100 facilitates user ownership, transparency, and control over personal dental health records, offering options for users to monetize the oral health data, and the clinical-grade data. Additionally, the system 100 integrates the subscriber-controlled digital wallet to ensure guaranteed oral health services. By leveraging proactive, data-driven preventive care strategies, the system 100 minimizes the necessity for costly dental treatments. Furthermore, the system 100 incentivizes user compliance through a rewards mechanism that issues tokens.

In one embodiment, the system 100 is optimized for streamlined lead generation, offering detailed clinical insights into prospective patients. The system 100 enables advanced scheduling capabilities, allowing the selection of specific conditions or treatments to prioritize. Additionally, the system 100 provides access to anonymized, high-quality clinical data, which can be utilized for research purposes or product development initiatives.

In one embodiment, the system 100 fundamentally transforms the paradigm of dental care from a reactive, therapeutic model to a proactive, data-driven, and preventive approach, while establishing a patient-centric marketplace for oral health data. By integrating blockchain technology, AI-driven analytics, and gamified incentive mechanisms, the system 100 creates an ecosystem that delivers value to subscribers and redefines the dental industry. The system 100 shifts away from the traditional costly, reactive model, fostering a preventive, patient-focused, and data-oriented framework that benefits subscribers, dental professionals, and the broader industry.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principles of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

It will readily be apparent that numerous modifications and alterations can be made to the processes described in the foregoing examples without departing from the principles underlying the invention, and all such modifications and alterations are intended to be embraced by this application.

The claimed invention is:

1. A system for managing and monetizing oral health data on a blockchain-based platform, comprising:
a computing device having a processor and a memory for storing one or more instructions executable by the processor, wherein the computing device is in communication with a server via a network,
wherein the processor is configured to:
enroll one or more subscribers in a dental plan through a subscription management module;
receive oral health data of the one or more subscribers associated with one or more dental activities through a data collection module;
analyze the oral health data to generate a custom dental health score through an artificial intelligence (AI) module, thereby determining schedule for one or more hygiene appointments to maintain oral health based on the custom dental health score;
obtain clinical-grade data of the one or more subscribers during the one or more hygiene appointments through a clinical data collection module;
enable the one or more subscribers to upload the clinical-grade data to a marketplace through a marketplace module;
enable the one or more subscribers to combine the oral health data with financial credit scores through the marketplace module to enhance a market value on the marketplace;
enable, by the marketplace module, the one or more subscribers to monetize and aggregate the oral health data into collective pools for group negotiations with one or more third-party service providers to secure desired pricing or dental plans, either individually or as a group;
issue, by a reward module, one or more tokens to the one or more subscribers upon performing at least one activity that comprises at least one of sharing the custom dental health score, and regularly attending the one or more hygiene appointments; and
store the collected oral health data, the clinical-grade data, and the custom dental health score of the one or more subscribers in one or more databases that are accessible through a subscriber-controlled digital wallet.

2. The system for managing and monetizing the oral health data of claim 1, wherein the data collection module comprises one or more dental devices that include at least one of a smart toothbrush attachment, a saliva testing kit, and a plaque scoring unit.

3. The system for managing and monetizing the oral health data of claim 1, wherein the oral health data comprises brushing data, saliva data, and plaque scoring data.

4. The system for managing and monetizing the oral health data of claim 1, wherein the clinical-grade data comprises at least one of intraoral radiographs, cone beam computed tomography (CBCT) scans, and three-dimensional (3D) intraoral scans, that is collected during the one or more hygiene appointments.

5. The system for managing and monetizing the oral health data of claim 1, wherein the one or more databases comprise at least one blockchain-enabled database.

6. The system for managing and monetizing the oral health data of claim 1, wherein the reward module is configured to:
generate new tokens based on compliance with the dental plan, and
enable trading of the one or more tokens on a cryptocurrency exchange, providing liquidity to the one or more subscribers.

7. The system for managing and monetizing the oral health data of claim 1, wherein the AI module is configured to provide recommendations for personalized oral hygiene products based on the custom dental health score.

8. The system for managing and monetizing the oral health data of claim 1, wherein the server is configured to execute gamified compliance programs, awarding the one or more tokens to the one or more subscribers for maintaining healthy dental metrics.

9. The system for managing and monetizing the oral health data of claim 1, wherein the marketplace module is configured enable the one or more subscribers to select at least one of specific buyers and industries for monetizing the collected oral health data, and the clinical-grade data.

10. The system for managing and monetizing the oral health data of claim 1, wherein the one or more tokens are configured to be used for discounts on dental care and related products within the blockchain-based platform.

11. The system for managing and monetizing the oral health data of claim 1, wherein the subscriber-controlled digital wallet is configured to integrate a visualization dashboard that displays a subscriber's oral health progress and compliance history.

12. A method for managing and monetizing oral health data using a system, comprising:
receiving, from a data collection module, oral health data of one or more subscribers associated with one or more dental activities;
analyzing, by an artificial intelligence (AI) module, the oral health data to generate a custom dental health score, thereby determining schedule for one or more hygiene appointments to maintain oral health based on the custom dental health score;
obtaining, by a clinical data collection module, clinical-grade data of the one or more subscribers during the one or more hygiene appointments;
enabling, by a marketplace module, the one or more subscribers to upload the clinical-grade data to a marketplace;
enabling, by the marketplace module, the one or more subscribers to combine the oral health data with financial credit scores to enhance a market value on the marketplace;
enabling, by the marketplace module, the one or more subscribers to monetize and aggregate the oral health data into collective pools for group negotiations with one or more third-party service providers to secure desired pricing or dental plans, either individually or as a group;
issuing, by a reward module, one or more tokens to the one or more subscribers upon performing at least one activity that comprises at least one of sharing the custom dental health score, and regularly attending the one or more hygiene appointments; and
storing the collected oral health data, the clinical-grade data, and the custom dental health score in one or more databases accessible through a subscriber-controlled digital wallet.

13. The method for managing and monetizing the oral health data of claim 12, wherein the data collection module comprises one or more dental devices that include at least one of a smart toothbrush attachment, a saliva testing kit, and a plaque scoring unit.

14. The method for managing and monetizing the oral health data of claim 12, wherein the oral health data comprises brushing data, saliva data, and plaque scoring data.

15. The method for managing and monetizing the oral health data of claim 12, wherein the clinical-grade data comprises at least one of intraoral radiographs, cone beam computed tomography (CBCT) scans, and three-dimensional (3D) intraoral scans, that is collected during the one or more hygiene appointments.

16. The method for managing and monetizing the oral health data of claim 12, wherein the one or more databases comprise at least one blockchain-enabled database.

17. The method for managing and monetizing the oral health data of claim 12, wherein the AI module is configured to provide recommendations for personalized oral hygiene products based on the custom dental health score.

18. The method for managing and monetizing the oral health data of claim 12, wherein the marketplace module is configured enable the one or more subscribers to select at least one of specific buyers and industries for monetizing the collected oral health data, and the clinical-grade data.

19. The method for managing and monetizing the oral health data of claim 12, wherein the one or more tokens are configured to be used for discounts on dental care and related products within the blockchain-based platform.

20. The method for managing and monetizing the oral health data of claim 12, wherein the subscriber-controlled digital wallet is configured to integrate a visualization dashboard that displays a subscriber's oral health progress and compliance history.
